SEQUENCE LISTING (48 sequences)

SEQ ID NO: 1 - protein, 458 aa, Homo sapiens
MEKTCIDALPLTMNSSEKQETVCIFGTGDFGRSLGLKMLQCGYSVVFGSRNPQKTTLLPSGAEVLSYSEAAKKSGIIIIAIHREHYDFLTELTEVLNGKILVDISNNLKINQYPESNAEYLAHLVPGAHVVKAFNTISAWALQSGALDASRQVFVCGNDSKAKQRVMDIVRNLGLTPMDQGSLMAAKEIEKYPLQLFPMWRFPFYLSAVLCVFLFFYCVIRDVIYPYVYEKKDNTFRMAISIPNRIFPITALTLLALVYLPGVIAAILQLYRGTKYRFPDWLDHWMLCRKQLGLVALGFAFLHVLYTLVIPIRYYVRWRLGNLTVTQAILKKENPFSTSSAWLSDSYVALGILGFFLFVLLGITSLPSVSNAVNWREFRFVQSKLGYLTLILCTAHTLVYGGKRFLSPSNLRWYLPAAYVLGLIIPCTVLVIKFVLIMPCVDNTLTRIRQGWERNSKH

SEQ ID NO: 2 - DNA, 4488 bp, Homo sapiens
agcgagagccacaagccacagcgctgagctgcaggcgcggcgaaacttccctctacccgcccggcccgcggcgcgcaccgttggcgctggacgcttcctccttggaagcgcctctccctcagttatggagaaaacttgtatagatgcacttcctcttactatgaattcttcagaaaagcaagagactgtatgtatttttggaactggtgattttggaagatcactgggattgaaaatgctccagtgtggttattctgttgtttttggaagtcgaaacccccagaagaccaccctactgcccagtggtgcagaagtcttgagctattcagaagcagccaagaagtctggcatcataatcatagcaatccacagagagcattatgattttctcacagaattaactgaggttctcaatggaaaaatattggtagacatcagcaacaacctcaaaatcaatcaatatccagaatctaatgcagagtaccttgctcatttggtgccaggagcccacgtggtaaaagcatttaacaccatctcagcctgggctctccagtcaggagcactggatgcaagtcggcaggtgtttgtgtgtggaaatgacagcaaagccaagcaaagagtgatggatattgttcgtaatcttggacttactccaatggatcaaggatcactcatggcagccaaagaaattgaaaagtaccccctgcagctatttccaatgtggaggttccccttctatttgtctgctgtgctgtgtgtcttcttgtttttctattgtgttataagagacgtaatctacccttatgtttatgaaaagaaagataatacatttcgtatggctatttccattccaaatcgtatctttccaataacagcacttacactgcttgctttggtttacctccctggtgttattgctgccattctacaactgtaccgaggcacaaaataccgtcgattcccagactggcttgaccactggatgctttgccgaaagcagcttggcttggtagctctgggatttgccttccttcatgtcctctacacacttgtgattcctattcgatattatgtacgatggagattgggaaacttaaccgttacccaggcaatactcaagaaggagaatccatttagcacctcctcagcctggctcagtgattcatatgtggctttgggaatacttgggttttttctgtttgtactcttgggaatcacttctttgccatctgttagcaatgcagtcaactggagagagttccgatttgtccagtccaaactgggttatttgaccctgatcttgtgtacagcccacaccctggtgtacggtgggaagagattcctcagcccttcaaatctcagatggtatcttcctgcagcctacgtgttagggcttatcattccttgcactgtgctggtgatcaagtttgtcctaatcatgccatgtgtagacaacacccttacaaggatccgccagggctgggaaaggaactcaaaacactagaaaaagcattgaatggaaaatcaatatttaaaacaaagttcaatttagctggatttctgaactatggttttgaatgtttaaagaagaatgatgggtacagttaggaaagtttttttcttacaccgtgactgagggaaacattgcttgtctttgagaaattgactgacatactggaagagaacaccattttatctcaggttagtgaagaatcagtgcaggtccctgactcttattttcccagaggccatggagctgagattgagactagccttgtggtttcacactaaagagtttccttgttatgggcaacatgcatgacctaatgtcttgcaaaatccaatagaagtattgcagcttccttctctggctcaagggctgagttaagtgaaaggaaaaacagcacaatggtgaccactgataaaggctttattaggtatatctgaggaagtgggtcacatgaaatgtaaaaagggaatgaggtttttgttgttttttggaagtaaaggcaaacataaatattaccatgatgaattctagtgaaatgaccccttgactttgcttttcttaatacagatatttactgagaggaactatttttataacacaagaaaaatttacaattgattaaaagtatccatgtcttggatacatacgtatctatagagctggcatgtaattcttcctctataaagaataggtataggaaagactgaataaaaatggagggatatccccttggatttcacttgcattgtgcaataagcaaagaagggttgataaaagttcttgatcaaaaagttcaaagaaaccagaattttagacagcaagctaaataaatattgtaaaattgcactatattaggttaagtattatttaggtattataatatgctttgtaaattttatattccaaatattgctcaatatttttcatctattaaattaatttctagtgtaaataagtagcttctatatctgtcttagtctattataattgtaaggagtaaaattaaatgaatagtctgcaggtataaatttgaacaatgcatagatgatcgaaaattacggaaaatcatagggcagagaggtgtgaagattcatcattatgtgaaatttggatctttctcaaatccttgctgaaatttaggatggttctcactgtttttctgtgctgatagtaccctttccaaggtgaccttcagggggattaaccttcctagctcaagcaatgagctaaaaggagccttatgcatgatcttcccacatatcaaaataactaaaaggcactgagtttggcatttttctgcctgctctgctaagacctttttttttttttactttcattataacatattatacatgacattatacaaaaatgattaaaatatattaaaacaacatcaacaatccaggatatttttctataaaactttttaaaaataattgtatctatatattcaattttacatcctttttcaaaggctttgtttttctaaaggctttgttttcctttttattatttttttcttttttatttttttgagacagtcttgctctgtcgctcaggctggagtgcagtggcacgatctcagctcactgcaacctcctcctcccaggttcaagtgattcttgttcatcagcctcccgagtagctgggactacaggcatgtgccactatgcccagctaatttttgtacttttagtagagacagggtttcaccacattggtcaggctggtcttgaaatgctggcgtcaagtgatctgcctgcctccgcctcacaaagcactgggattacaggcatgaatctggccttacgtaatatattttcttaatggctgcataatatcacatcaaataggcatttttcaaacctctttccttattaaacatgtagactatatccattttttactaaaataaataacatttcagataatatctttgcactgataatgttgccaagccatttctaaagtgaccttatcaatttaattaccattggatgagggtgttgctttcatcgcaccattgtagattgtcttttttatttcaatttgcgtttatttataactggttgcaaaggtacacagaacacacgctccttcaacttatctttgataaacccaagcaaggatacaaaaagttggacgacattgagtagagtcatggtatacggtgctgaccctacagtatcagtggaaaagataaggaaaatgtcactactcacctatgttatgcaaaacagttaggtgtgctggggctggatactgctcttttacttgagcattggttgattaaagtttaggtaccatccaggctggtctagagaagtctttggagttaaccatgctctttttgttaaagaagagagtaatgtgtttatcctggctcatagtccgtcaccgaaaatagaaaatgccatccataggtaaaatgctgacctatagaaaaaaatgaactctacttttatagcctagtaaaaatgctctacctgagtagttaaaagcaattcatgaagcctgaagctaaagagcactctgatggttttggcataatagctgcatttccagacctgacctttggccccaaccacaagtgctccaagccccaccagctgaccaaagaaagcccaagttctccttctgtccttcccacaacctccctgctcccaaaactatgaaattaatttgaccatattaacacagctgactcctccagtttacttaaggtagaaagaatgagtttacaacagatgaaaataagtgctttgggcgaactgtattccttttaacagatccaaactattttacatttaaaaaaaaagttaaactaaacttctttactgctgatatgtttcctgtattctagaaaaatttttacactttcacattatttttgtacactttccccatgttaagggatgatggcttttataaatgtgtattcattaaatgttactttaaaaataaaaaaaaaaaaaaaa

SEQ ID NO: 3 - protein, 470 aa, Mus musculus
MEKAHADEFPLTTDSSEKQGVVCIFGTGDFGKSLGLKMLQCGYSIVFGSRNPQVSSLLPRGAEVLSYSEAASKSDIIILAMHREHYDSLTELVDYLKGKVLVDVSNNRKINQYPESNAEYLAQLEPGAHVVKAFNTISAWALQSGTLDASRQVFVCGNDSKAKQRVMDIARTLGLTPLDQGSLMAASEIENYPLQLFPMWRFPFYLSSVLCVFFFVYCAIREVIYPYVNGKTDATYRLAISIPNRVFPITALILLALVYLPGILAAILQLYRGTKYRRFPNWLDHWMLCRKQLGLVALGFAFLHVIYTLVIPIRYYVRWRLRNATITQALTNKDSPFITSYAWINDSYLALGILGFFLFLLLGITSLPSVSNMVNWREFRFVQSKLGYLTLVLCTAHTLVYGGKRFLSPSILRWSLPSAYILALVIPCAVLVLKCILIMPCIDKTLTRIRQGWERNSKYTQSALNGKSDI

SEQ ID NO: 4 - DNA, 3140 bp, Mus musculus
ctgcaagcctagaaggcagagcccaccgcagccgcgccagaccaacgcccctccgggcgcctcgccgcagtaatggagaaagcacatgcagatgagtttcctctgactactgattcctcagaaaagcaaggggttgtctgcattttcggaacaggagatttcgggaagtcactgggattgaaaatgcttcagtgtggctattctatcgtttttgggagtcgaaacccccaggtgtccagccttctgcccaggggggcagaggtcctgagctattcggaagcagcatccaagtctgacatcataatcctagccatgcacagagagcactatgattccctcacggaactagttgattatctcaaaggaaaagtattggtagacgtcagtaacaaccgcaaaatcaatcagtatccagagtcaaatgcggaataccttgctcagctggagccaggagcccacgtggtcaaagcatttaacaccatctcagcctgggctctccagtcaggaacactagatgcaagccggcaggtgtttgtctgtggaaatgacagcaaggccaaacaaagagtaatggacattgctcgtactcttgggctcaccccactggaccaaggatctctcatggcagccagtgaaattgaaaactaccctctgcaactatttccaatgtggaggtttcctttctatttgtcctctgtgctgtgcgtcttcttctttgtgtactgtgcaatacgagaggtgatatacccctacgtgaatgggaaaacagatgctacgtaccgcctggccatttccatcccgaatcgtgtctttcctataacagcactgatcctgcttgccttggtgtacctccctggtattctcgctgccattctgcagctctatcgaggcacaaagtaccgccgattcccaaactggcttgaccattggatgctttgcagaaagcaactgggcttggtagctctgggatttgccttccttcatgtcatctacacacttgtgattcctatccgttactatgttcgatggaggctgagaaatgcaaccattactcaggccctaactaataaagacagcccatttattacctcttatgcctggattaatgattcctacttggcgctgggaatcctgggattttttctgttccttctgttgggaatcacttccttgccatcagtaagcaacatggtcaactggagagagttccggtttgttcagtccaaactgggttatctgaccctggtcttgtgcacagcccacactttggtgtatggcggaaagagattcctcagcccttcgattctaaggtggagcctcccttcggcttatattttggcactcgtcatcccctgcgcagtgctggtgctgaagtgcatcctcatcatgccatgcatagacaagaccctcactcgcatccgccagggctgggaaagaaactcaaagtacacacaatcagcactgaacggaaaatcggatatttaagctacagttgaatttgccaggatttgtgtactattgcagtaaatgtttagagcataatgtgtccagttataagaaagcattttctcttgtcatggccctaactttatcatcacagtaggaagtgttgcctcactttgaaagaccagaatagcaagggaaccctcgctgcctcagtcacgtgatgactcattgtgggcctccctcacttcctttcccagaaaccccagggatgccactgtgattagatttacagttccacacagcgaagcttcttgattatgggcaagttgaatgacgcaatgttttgtagagttctggaagagtgtgtgcaacttccttccctaattcaagggctgatgtaagtgaaaaagcagcactcggcatactacaggttttgcttggcatgcctggagaaaagccccaaatgaaatagacagaaagagggttagtttgtttcctggaagaagtggtgatcataactattgtcatactgatctgggggtatttggggtttagctgtagtgctgagctcaaacctaaggccttatgcatgttaggtaaatgatatgttactgagccacacacacgccagccgtgccatagtgagtatgtagtcatatgtacccccttgatttcttgcttttaccaatatggattcttactatggggaaatattttatactgcaacaaaattggtttgaagtagctgttccttggatatatggaaagctatgtaattgtcccaagattcttcctccacaaaggacggcttgaggggagatggggtaagagttgagcagcgatcctaagttgagggaagatggggaaagagtggaagatccattgggtttttcaagtgccgcaggcagacacaggctgataatagccttgtacaaaaatgtcagataagccagaattttagctagtaagctacgcaaatactaatacagtttcactatgctgtttgggatcatttaggagtggtggtgtgctttcttcattatacatttcaaatactgttcagtattaagcatctgtgaaattaacttctagtacaaacatgtagcttccataatctgtctttgttatcaatgtaagtggtgcagttagaaggaatcatctgaactcgtcggagcgaggaaaggatagctgagagaactgtggagtgctagggtgtaggcgagcagcagtggaccatgggtcggatgcagccagctctggcttcctgtgtggacagtgcctttcccaaggtgaccttcagcaggctgagaatccttagctctagcctggagctaaaaggagatctttgcacaatcttcccgcgtgtcactgaaataaagaaaaggcattgagtttgacatttttctgattaatctacaaagactgtttatttttcatttttctagcttttatatcgtatattgtacatgatatcatacattgctcacaatccagggttttttatataaagctcttatataacacagacatttcttttactattgccctaataaaacatatagagtatattcagtttttactaagaggacaattgaggtaatgtctttgtactaacaaaatatttctgtattttcaattatttccttggaatattttccaagagttagtataaaattaaagaatatttgataaca

SEQ ID NO: 5 - DNA, 21 bp, Artificial Sequence
  Primer
gctgctcggatcactagtgaa

SEQ ID NO: 6 - DNA, 21 bp, Artificial Sequence
  Primer
ttctgctatcagtctgtccag

SEQ ID NO: 7 - DNA, 22 bp, Artificial Sequence
  Primer
aaccttagatgggggtgtcctg

SEQ ID NO: 8 - DNA, 20 bp, Artificial Sequence
  Primer
tcgtggaagtgacgcctttc

SEQ ID NO: 9 - DNA, 20 bp, Artificial Sequence
  Primer
atgtaacccaggacgctgag

SEQ ID NO: 10 - DNA, 20 bp, Artificial Sequence
  Primer
gtcgcagtgactttcccaat

SEQ ID NO: 11 - DNA, 18 bp, Artificial Sequence
  Primer
gaaactgcaggagctgtc

SEQ ID NO: 12 - DNA, 18 bp, Artificial Sequence
  Primer
cacggagttgaggcggat

SEQ ID NO: 13 - DNA, 18 bp, Artificial Sequence
  Primer
atggtggacacggaaagc

SEQ ID NO: 14 - DNA, 18 bp, Artificial Sequence
  Primer
ttaatcgtcctctacgac

SEQ ID NO: 15 - DNA, 18 bp, Artificial Sequence
  Primer
gaaatgaccatggttgac

SEQ ID NO: 16 - DNA, 18 bp, Artificial Sequence
  Primer
gatgcaggctccactttg

SEQ ID NO: 17 - DNA, 21 bp, Artificial Sequence
  Primer
atgggtgaaactctgggagat

SEQ ID NO: 18 - DNA, 18 bp, Artificial Sequence
  Primer
gatgcaggctccactttg

SEQ ID NO: 19 - DNA, 25 bp, Artificial Sequence
  Primer
cctctacttggaagacgacattcgc

SEQ ID NO: 20 - DNA, 24 bp, Artificial Sequence
  Primer
gcagccgagctttgtaagagcggt

SEQ ID NO: 21 - DNA, 28 bp, Artificial Sequence
  Primer
acggcagcccctgtaacgaccactgtga

SEQ ID NO: 22 - DNA, 28 bp, Artificial Sequence
  Primer
tgccaagatggttccgccactcaccagg

SEQ ID NO: 23 - DNA, 18 bp, Artificial Sequence
  Primer
gactacctcatgaagatc

SEQ ID NO: 24 - DNA, 18 bp, Artificial Sequence
  Primer
gatccacatctgctggaa

SEQ ID NO: 25 - DNA, 20 bp, Artificial Sequence
  Primer
gaacggccactacgacaaat

SEQ ID NO: 26 - DNA, 20 bp, Artificial Sequence
  Primer
ctgcaggttctcaatgcaaa

SEQ ID NO: 27 - DNA, 20 bp, Artificial Sequence
  Primer
tcacctggaagacagctcct

SEQ ID NO: 28 - DNA, 20 bp, Artificial Sequence
  Primer
tcgactttccatcccacttc

SEQ ID NO: 29 - DNA, 24 bp, Artificial Sequence
  Primer
tcctctgacatttgcaggtctatc

SEQ ID NO: 30 - DNA, 20 bp, Artificial Sequence
  Primer
aaaggcattggctggaagaa

SEQ ID NO: 31 - DNA, 20 bp, Artificial Sequence
  Primer
gctgtgcttgcagcttactg

SEQ ID NO: 32 - DNA, 20 bp, Artificial Sequence
  Primer
cggatcaccttcttgagagc

SEQ ID NO: 33 - DNA, 20 bp, Artificial Sequence
  Primer
gcagctcgtacaggtcatca

SEQ ID NO: 34 - DNA, 20 bp, Artificial Sequence
  Primer
actgccgttgtctgtcactg

SEQ ID NO: 35 - DNA, 21 bp, Artificial Sequence
  Primer
tctccatgacagacatggaca

SEQ ID NO: 36 - DNA, 20 bp, Artificial Sequence
  Primer
gtcaggctgttggtctcaca

SEQ ID NO: 37 - DNA, 23 bp, Artificial Sequence
  Primer
gggtgaaactctgggagattctc

SEQ ID NO: 38 - DNA, 19 bp, Artificial Sequence
  Primer
tcagcaaccattgggtcag

SEQ ID NO: 39 - DNA, 20 bp, Artificial Sequence
  Primer
ttcttacacgaccaccacca

SEQ ID NO: 40 - DNA, 20 bp, Artificial Sequence
  Primer
cagccgagccttgtaagttc

SEQ ID NO: 41 - DNA, 30 bp, Artificial Sequence
  Primer
atctgttgtaaggtgtatttgctggcttgg

SEQ ID NO: 42 - DNA, 30 bp, Artificial Sequence
  Primer
cttcgcctatgctggtgcacagagatgact

SEQ ID NO: 43 - DNA, 20 bp, Artificial Sequence
  Primer
accacagtccatgccatcac

SEQ ID NO: 44 - DNA, 20 bp, Artificial Sequence
  Primer
tccaccaccctgttgctgta

SEQ ID NO: 45 - DNA, 23 bp, Artificial Sequence
  Primer
ggagtacatgaagatggacctgg

SEQ ID NO: 46 - DNA, 21 bp, Artificial Sequence
  Primer
ctgttcgcatgtcagcatagc

SEQ ID NO: 47 - DNA, 18 bp, Artificial Sequence
  Primer
cgaaacttccctctaccc

SEQ ID NO: 48 - DNA, 18 bp, Artificial Sequence
  Primer
acacaaacacctgccgac

(19) United States
(12) Patent Application Publication
(10) Pub. No.: US 2016/0271210 A1
(43) Pub. Date: Sep. 22, 2016

(54) METHOD FOR TREATING OR PREVENTING NONALCOHOLIC FATTY LIVER DISEASE

(71) Applicant: Dong-A University Research Foundation for Industry- Academy Cooperation, Busan (KR)

(72) Inventors: Young Hyun YOO, Busan (KR); Hye Young KIM, Ulsan (KR)

(21) Appl. No.: 14/662,214

(22) Filed: Mar. 18, 2015

(51) Int. Cl. A61K 38/17; A61K 9/00; C12N 7/00; A61K 31/713

(52) U.S. Cl. CPC A61K 38/1703; A61K 31/713; A61K 9/0019; C12N 7/00; A61K 48/00

(57) ABSTRACT

A method for treating or preventing non-alcoholic fatty liver disease is disclosed. By promoting the expression of six-transmembrane protein of prostate 2 (STAMP2) in liver cells, the method can be useful in improving the abnormalities in lipid metabolism in the liver, and also improving insulin resistance, thereby preventing or treating a non-alcoholic fatty liver disease.

### FIELD OF THE INVENTION

The invention relates to a method for treating or preventing non-alcoholic fatty liver disease.

### BACKGROUND OF THE INVENTION

Irrespective of lively studies, our understanding of the pathogenesis of non-alcoholic fatty liver disease (NAFLD) remains incomplete. Employment of animal models has proposed the “two-hit” hypothesis for the pathogenesis of NAFLD. The “first hit” is accumulation of lipid in the liver. Thus, disruption of the normal mechanisms for synthesis, transport and removal of free fatty acids (FFAs) and triglycerides is the primary basis for the development of NAFLD. The “first hit” is followed by a “second hit” in which proinflammatory mediators induce inflammation, hepatocellular injury and fibrosis. This hypothesis has recently replaced by more complex model. Non-glyceride fatty acids metabolites were demonstrated to play a central role in lipotoxicity and the pathogenesis of steatohepatitis. Metabolic oxidative stress, autophagy and inflammation are indicated to be hallmarks of non alcoholic steatohepatitis (NASH) progression. This inflammatory state of NASH may result in the deposition of fibrous tissue, including but not limited to collagen, which can lead to cirrhosis, nodule formation, and eventually hepatocellular carcinoma.

An excessive amount of intrahepatic triglyceride in NAFLD results from an imbalance of complex interactions of metablic events. Players participating in increased fatty acid synthesis include sterol regulatory element-binding protein-1c (SREBP-1c), fatty acid synthase (FAS), peroxisome proliferator-activated receptor γ (PPARγ), hepatic stearoyl-CoA desaturase 1 (SCD1), adipocyte fatty acid-binding protein (A-FABP; also known as FABP-4 or aP2) and acetyl coenzyme A carboxylase-1 (ACC1). Aberrant induction of these factors may contribute to hepatic steatosis. Insulin resistance and steatosis is closely associated in the development of NAFLD. Phosphatidylinositol 3-kinase (PI3K), Akt and insulin receptor substrate (IRS)-1, and their phosphorylation plays a pivotal role in insulin signaling pathway. Fatty acid synthesis cross-talks with insulin signaling. Increased adiposity and insulin resistance leads to elevated circulating level of FFAs in NAFLD patients. Elevated hepatic FFAs worsen insulin resistance mediated via lipid intermediates which are a significant contributor to the insulin resistance. Hypersinsulinemia also activates SREBP-1c. Insulin activates the PI3K/Akt pathway which is responsible for insulin stimulated glucose uptake. PI3K, in response to insulin, leads to the phosphorylation of Akt. Then, phosphorylated Akt induces GLUT4 translocation to the plasma membrane which directly increases glucose transport into cells.

NAFLD treatments that primarily target the main components of metabolic syndrome have focused on the insulin resistance, oxidative stress, proinflammatory cytokines and bacterial overgrowth involved in NAFLD pathogenesis. However, currently, no drug is available for NAFLD treatment.

Six-transmembrane protein of prostate 2 (STAMP2) as known as six-transmembrane epithelial antigen of prostate 4 (STEAP4) belongs to the STEAP protein family.

In the previous studies on STAMP2, it was found that STAMP2 in fat cells is a main regulator in the inflammatory response and metabolism, and insulin resistance, abnormalities in lipid metabolism, fatty liver diseases, and the like are induced by deletion of STAMP2 from the fat cells (Wellen K E, Fucho R, Gregor M F, Furuhashi M, Morgan C, Lindstad T et al. Coordinated regulation of nutrient and inflammatory responses by STAMP2 is essential for metabolic homeostasis. Cell 2007; 129:537-548.). Therefore, STAMP2 in the fat cells is known to be associated with the lipid and insulin metabolisms. However, this study shows the roles of STAMP2 in the fat cells, and thus the roles of STAMP2 in the liver, and the relationship between an increase in expression of STAMP2 and treatment of disease symptoms in the liver are not easily deducible therefrom.

### SUMMARY

This invention discloses a method for treating or preventing a non-alcoholic fatty liver disease, comprising a step of promoting the expression of STAMP2 (Six-transmembrane protein of prostate 2) in a liver cell.

In some embodiments of the present invention, the method may further include a step of administering a vehicle, into which a gene encoding STAMP2 is introduced, to a subject before the step of promoting the expression of STAMP2.

In some embodiments of the present invention, the vehicle is an adenovirus.

In some embodiments of the present invention, the administration may be intravenous administration.

In some embodiments of the present invention, the vehicle may be administered at a dose of 1×10⁸ to 1×10¹¹ plaque-forming units (pfus).

In some embodiments of the present invention, the method may further include a step of inhibiting expression of at least one protein selected from the group consisting of ACC1, FAS, SCD1, SREBP1, aP2, CD36, and PPARγ in liver cells after the step of promoting the expression of STAMP2.

In some embodiments of the present invention, the method may further include a step of inhibiting degradation of IRS1 in the liver cells after the step of promoting the expression of STAMP2.

In some embodiments of the present invention, the non-alcoholic fatty liver disease is non-alcoholic simple steatosis, non-alcoholic steatohepatitis, non-alcoholic steatohepatitis with liver fibrosis, non-alcoholic steatohepatitis with cirrhosis, or non-alcoholic steatohepatitis with cirrhosis and hepatocellular carcinoma.

This invention discloses a method to treat a subject having a non-alcoholic fatty liver disease, comprising a step of administrating an effective amount of a vehicle, into which a gene encoding STAMP2 is introduced, to the subject.

In some embodiments of the present invention, the vehicle is an adenovirus.

In some embodiments of the present invention, the administration may be intravenous administration.

In some embodiments of the present invention, the vehicle may be administered at a dose of 1×10⁸ to 1×10¹¹ plaque-forming units (pfus).

In some embodiments of the present invention, the method may further include a step of inhibiting expression of at least one protein selected from the group consisting of ACC1, FAS, SCD1, SREBP1, aP2, CD36, and PPARγ in liver cells after the step of promoting the expression of STAMP2.

In some embodiments of the present invention, the method may further include a step of inhibiting degradation of IRS1 in the liver cells after the step of promoting the expression of STAMP2.

In some embodiments of the present invention, the non-alcoholic fatty liver disease is non-alcoholic simple steatosis, non-alcoholic steatohepatitis, non-alcoholic steatohepatitis with liver fibrosis, non-alcoholic steatohepatitis with cirrhosis, or non-alcoholic steatohepatitis with cirrhosis and hepatocellular carcinoma.

This invention discloses a pharmaceutical composition for treatment or prevention of a non-alcoholic fatty liver disease, comprising a vehicle, into which a gene encoding STAMP2 is introduced.

In some embodiments of the present invention, the vehicle may be an adenovirus.

In some embodiments of the present invention, the vehicle may be included at a dose of 1×10⁸ to 1×10¹¹ plaque-forming units (pfus).

In some embodiments of the present invention, the non-alcoholic fatty liver disease is non-alcoholic simple steatosis, non-alcoholic steatohepatitis, non-alcoholic steatohepatitis with liver fibrosis, non-alcoholic steatohepatitis with cirrhosis, or non-alcoholic steatohepatitis with cirrhosis and hepatocellular carcinoma.

The above objectives and advantages of the present invention will become more readily apparent to those ordinarily skilled in the art after reviewing the following detailed descriptions and accompanying drawings.

### BRIEF DESCRIPTION OF THE DRAWINGS

FIG. 1. STAMP2 expression is markedly reduced in livers obtained from human NAFLD patients and NAFLD mice. (A) Expression of STAMP2 was markedly reduced in hepatocytes from non-alcoholic steatosis patient. (B) Expression of STAMP2 was markedly reduced in hepatocytes of mice fed an HFD for 22 weeks. (C) Expression of STAMP2 was markedly reduced in hepatocytes of mice fed an HFD for 15 weeks. (D) RT-PCR and western blot analyses showed that the hepatic expression of STAMP2 was markedly reduced in mice fed an HFD for 15 weeks. n=6 per group. *, P<0.05 and **, P<0.01 compared with mice fed an SD.

FIG. 2. Liver specific deletion of STAMP2 accelerates hepatic steatosis and insulin resistance in HFD-induced NAFLD mice. siS2, siSTAMP2; siF, siFVII. (A) siSTAMP2 efficiently downregulated STAMP2 mRNA and protein expression. (B) Body weight was significantly higher in siSTAMP2-injected mice. n=7-10 per group. (C) siSTAMP2 markedly induced the vacuolization. (D) Liver weight and plasma TC, TG and NEFA levels were significantly higher in siSTAMP2-injected mice. n=7-10 per group. (E) Plasma insulin level was significantly higher in siSTAMP2-injected mice. n=7-10 per group. (F) The blood glucose levels over the entire time course of the GTT and ITT were significantly higher in the siSTAMP2-injected mice. n=7-10 per group. *, P<0.05 and **, P<0.01 compared with the siFVII experimental controls.

FIG. 3. Adenoviral overexpression of STAMP2 improves hepatic steatosis in HFD24 induced NAFLD mice. (A) Ad-STAMP2 efficiently increased STAMP2 mRNA and protein expression. (B) Ad-STAMP2 significantly decreased body weight. 1 n=10-12 per group. *, P<0.05 and **, P<0.01 compared with the Ad-Empty experimental control at the same time point. (C) Ad-STAMP2 markedly attenuated HFD-induced vacuolization. (D) Ad-STAMP2 significantly decreased the liver weight. Ad-E, Ad-Empty; Ad-S2, Ad-STAMP2. n=10-12 per group. (E) Ad-STAMP2 significantly attenuated the HFD-induced increase of plasma TC, TG and NEFA levels. n=10-12 per group. *, P<0.05 and **, P<0.01 compared with the experimental control.

FIG. 4. Hepatic STAMP2 downregulates lipogenic and adipogenic factors in vivo and in vitro. (A) Ad-STAMP2 downregulated the expression of SREBP1 and PPARγ proteins. pSREBP1, premature SREBP1; mSREBP1, mature SREBP1. (B) RT-PCR showed that Ad-STAMP2 attenuated HFD-induced upregulation of lipogenic and adipogenic genes. n=8 per group. (C) Luciferase assay showed that Ad-STAMP2 attenuated OA-induced upregulation of the promoter activity of SREBP1c and PPARγ in vitro. n=5. (D) RT-PCR showed that Ad-STAMP2 attenuated the expression of OA-induced upregulation of lipogenic and adipogenic genes. n=4. (E) siSTAMP2 markedly augmented the protein levels of SREBP1 and PPARγ in mice fed an HFD. *, P<0.05 and **, P<0.01 compared with the experimental control.

FIG. 5. Adenoviral overexpression of STAMP2 improves insulin sensitivity and glucose metabolism in vivo and in vitro. (A&B) Ad-STAMP2(Ad-S2) attenuated the HFD-induced increase in the (A) blood glucose level and (B) plasma insulin level. n=10-12 per group. (C) Ad-STAMP2 reversed the HFD-induced impairment of the glucose and insulin tolerances. n=10-12 per group. (D) Ad-STAMP2 augmented the insulin-induced upregulation of p-Akt(S473) and p-PI3K p85(Y458) in vitro. (E) Ad-STAMP2 reversed the OA-induced upregulation of the mRNA expression levels of PEPCK and G6Pase in vitro. (F) Ad-STAMP2 augmented the insulin-induced suppression of the promoter activity of PECK and G6Pase in vitro. n=7. *, P<0.05 and **, P<0.01 compared with the experimental control.

FIG. 6. Hepatic STAMP2 prevents degradation of IRS1. (A) The protein level of IRS1 was reduced in the mice fed an HFD compared to the mice fed an SD. (B) In siSTAMP2-injected mice fed an HFD, the IRS1 protein level was reduced compared to the experimental controls. (C&D) Ad-STAMP2 reversed diet-induced reduction of the IRS1 protein level but not the mRNA level in vivo (C) and in vitro (D). (E) Ad-STAMP2 reversed the OA-stimulated serine phosphorylation of IRS1 in vitro. n=4. (F) The protein level of IRS1 accumulated by treatment with proteasome inhibitor MG132 (25 μM, 2 h) in vitro. **, P<0.01 compared with the experimental controls.

FIG. 7. HFD induces hepatic steatosis. (A) A representative photograph of the mice fed either an SD or a 60% HFD (15 weeks). Compared with mice on the SD, mice on the HFD were markedly heavier. Body weight was monitored weekly. n=6 per group. (B) A representative photograph of the livers obtained from mice fed either an SD or HFD. The average weights of the livers after the mice were fed an SD or HFD for 15 weeks are shown. n=6 per group. (C) Representative H & E staining of liver sections obtained from mice fed either an SD or HFD. Vacuolization and lipid accumulation are observed in the liver from mice fed HFD. (D) The average plasma cholesterol levels (mg/dl) after SD or HFD feeding for 15 weeks are illustrated. (E) Average plasma TG amounts (mg/dl) after SD or HFD feeding for 15 weeks are illustrated. (F) Average NEFA amounts (μEq/l) after SD or HFD feeding for 15 weeks are illustrated. The values represent the mean±SD, n=6 per group. *, p<0.05 and **, P<0.01 compared with the experimental control.

FIG. 8. HFD (15 weeks) induces insulin resistance in C57BL/6 mice. (A) Six to fifteen weeks after introducing the HFD, the blood glucose levels were markedly increased. n=6 per group. *, p<0.05 and **, p<0.01 compared with the SD group at the same time point. (B) The plasma insulin levels were increased in the mice fed an HFD for 15 weeks. n=6 per group. *, p<0.05 compared with the SD group. (C) Glucose tolerance was impaired in the mice fed an HFD for 15 weeks. n=6 per group. *, p<0.05 and **, p<0.01 compared with the SD group at the same time point. (D) Insulin tolerance was impaired in the mice fed an HFD for 15 weeks. The values represent the mean±SD, n=6 per group. *, p<0.05 and **, p<0.01 compared with the SD group at the same time point.

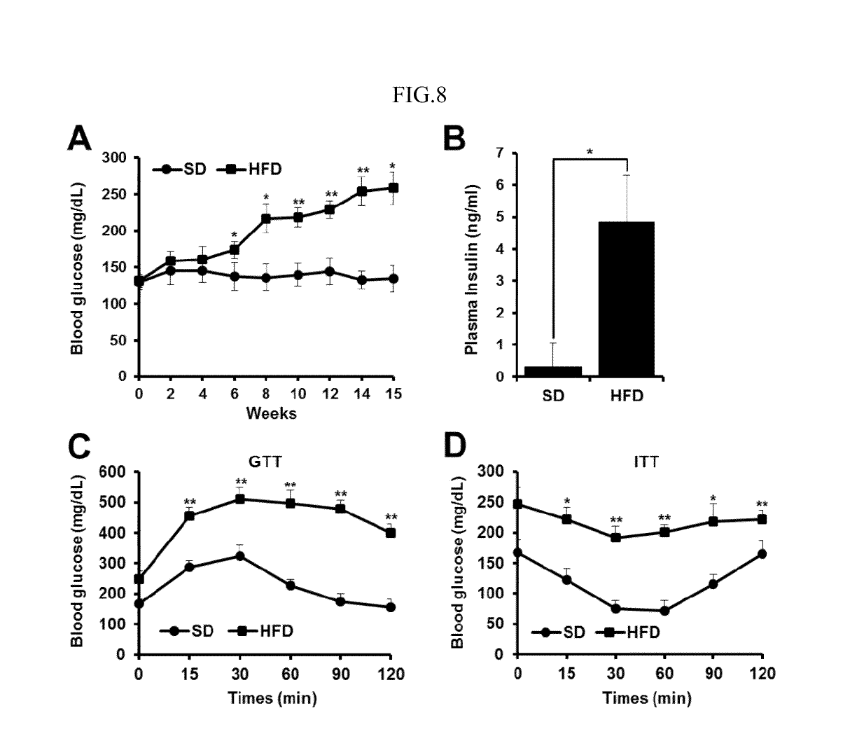

FIG. 9 STAMP2 expression in the liver, adipose and muscle. (A) siSTAMP2 was delivered to the livers of mice fed an HFD for 10 weeks. Ten days after the siSTAMP2 injection, the animals were sacrificed, and to confirm whether STAMP2 siRNA is specific for the liver, RT-PCR for STAMP2 was performed in adipose, muscle and liver (upper panel) Immunohistochemistry (lower panel) shows that most hepatocytes displaying vacuolization and lipid accumulation in the siSTAMP2-Invivofectamine 2.0 complex-injected mice were negative for STAMP2, whereas most hepatocytes in the siFVII-Invivofectamine 2.0 complex-injected mice that were fed the HFD were positive for STAMP2. siF, siFVII; siS, siSTAMP2. (B) Adenoviral STAMP2 (Ad-STAMP2) was delivered to the mice fed an HFD for 15 weeks. One week after the injection, the animals were sacrificed, and to confirm whether adenoviral empty vector or STAMP2 is specific for the liver, RT-PCR for adenovirus type 5 fiber protein was performed in adipose, muscle and liver (upper panel) Immunohistochemistry (lower panel) shows that most hepatocytes displaying vacuolization and lipid accumulation in the Ad-Empty-injected mice were negative for STAMP2. Most hepatocytes were not steatotic in the Ad-STAMP2-injected mice, which were positive for STAMP2. AdE, AdEmpty; AdS, AdSTAMP2.

FIG. 10. STAMP2 reduces the FFA-induced lipid accumulation in vitro. The effect of STAMP2 in FFA-induced lipid accumulation was tested in HepG2 cells. Three FFAs including oleic acid (OA or O), palmitic acid (PA or P) and stearic acid (SA or S) were tested. O/P and O/P/S are mixtures of FFAs. DMEM containing fatty acid-free BSA was used as the experimental control. (A) The three FFAs and two mixtures tested significantly increased the lipid accumulation in HepG2 cells. (B) Oil red O staining demonstrated that these FFAs induced lipid accumulation in HepG2 cells. (C) Silencing of the STAMP2 gene, which efficiently downregulated STAMP2 mRNA and protein, significantly augmented the FFA-induced lipid accumulation. (D) Ad-STAMP2, which efficiently overexpressed STAMP2 mRNA and protein, significantly reduced the FFA-induced lipid accumulation. Values represent the mean±SD *, P<0.05 and **, P<0.01 compared with the experimental control. All RT-PCR and Western blot assay data are representative of four independent experiments.

FIG. 11. STAMP2 regulates OA-induced lipid accumulation in vitro. (A) STAMP2 knockdown augmented the OA-induced lipid accumulation. (B) STAMP2 overexpression reversed the OA-induced lipid accumulation. All values represent the mean±SD, n=3. *, p<0.05 and **, p<0.01 compared with the experimental control.

FIG. 12. Primers Used for PCR Amplification

### DETAILED DESCRIPTION

The present invention will now be described more specifically with reference to the following embodiments. It is to be noted that the following descriptions of preferred embodiments of this invention are presented herein for purpose of illustration and description only; they are not intended to be exhaustive or to be limited to the precise form disclosed.

In some embodiments of the present invention, the method of the present invention includes a step of promoting the expression of six-transmembrane protein of prostate 2 (STAMP2) in liver cells.

By promoting the expression of STAMP2 in the liver cells, the method of the present invention may be used to improve the abnormalities in lipid metabolism in the liver, and also improve the insulin resistance, thereby preventing or treating a non-alcoholic fatty liver disease.

For example, the non-alcoholic fatty liver disease may include non-alcoholic simple steatosis, non-alcoholic steatohepatitis, non-alcoholic steatohepatitis with liver fibrosis, non-alcoholic steatohepatitis with cirrhosis, or non-alcoholic steatohepatitis with cirrhosis and hepatocellular carcinoma.

In still another embodiment of the present invention, the promotion of the expression of STAMP2 in the liver cells results in a decrease in expression of at least one protein selected from the group consisting of an acetyl coenzyme A carboxylase-1 (ACC1), a fatty acid synthase (FAS), a hepatic stearoyl-CoA desaturase 1 (SCD1), a sterol regulatory element-binding protein-1 (SREBP1), an adipocyte fatty-acid-binding protein (aP2), a cluster of differentiation 36 (CD36, also known as a fatty acid translocase (FAT)), and a peroxisome proliferator-activated receptor γ (PPARγ) in the liver cells.

The ACC1, FAS, SCD1, SREBP1, aP2, CD36, and PPARγ are lipogenic or adipogenic factors that participate in the synthesis of fatty acids. Therefore, according to one exemplary embodiment of the present invention, the non-alcoholic fatty liver disease may be prevented or treated by reducing the expression of these proteins in the liver cells to improve the abnormalities in lipid metabolism. This is done by reducing expression of genes encoding the lipogenic factor and the adipogenic factor.

In still another embodiment of the present invention, the promotion of the expression STAMP2 in the liver cells causes inhibition of degradation of an insulin receptor substrate-1 (IRS1) in the liver cells.

In an NAFLD animal model, an IRS1 level in the liver cells is reduced to cause insulin resistance. According to one exemplary embodiment of the present invention, the insulin resistance may be improved by inhibiting the degradation of IRS1. In still another embodiment of the present invention, the inhibition of the degradation of IRS1 may be performed by inhibiting phosphorylation of Ser307 of IRS1.

In still another embodiment of the present invention, the method may further include a step of administering a vehicle, into which a gene encoding STAMP2 is introduced, to a subject before the step of promoting the expression of STAMP2.

In still another embodiment of the present invention, the method of the present invention may include a step of administering an effective amount of a vehicle, into which a gene encoding STAMP2 is introduced, to the subject.

In yet another embodiment of the present invention, the pharmaceutical composition of the present invention may include the vehicle into which a gene encoding STAMP2 is introduced.

The term “subject” refers to an animal, preferably a mammal, and most preferably a human, who is the object of treatment, observation or experiment. The mammal may be selected from the group consisting of mice, rats, hamsters, gerbils, rabbits, guinea pigs, dogs, cats, sheep, goats, cows, horses, giraffes, platypuses, primates, such as monkeys, chimpanzees, and apes. In some embodiments, the subject is a human.

The term “effective amount” of a compound refers to a sufficient amount of the compound that provides a desired effect but with no- or acceptable-toxicity. This amount may vary from subject to subject, depending on the species, age, and physical condition of the subject, the severity of the disease that is being treated, the particular compound used, its mode of administration, and the like. A suitable effective amount may be determined by one of ordinary skill in the art.

The gene encoding STAMP2 is a gene encoding STAMP2 derived from the subject. In this case, genes known in the related art may be used as the gene encoding STAMP2. For example, Human STAMP2 may be used which have amino acid sequences of SEQ ID NO:1 and its coding gene(mRNA) may have nucleotide sequences of SEQ ID NO:2. Murine(Mus musculus) STAMP2 may be used which have amino acid sequences of SEQ ID NO: 3 and its coding gene(mRNA) may have nucleotide sequences of SEQ ID NO:4.

Vehicles known in the related art may be used without limitation as the vehicle delivering the gene encoding STAMP2, and may, for example, include a liposome, a plasmid vector, a cosmid vector, a bacteriophage vector, a viral vector, etc., and preferably a viral vector.

Specific examples of the viral vector may include an adenovirus, an adeno-associated virus, a retrovirus, a lentivirus, a herpes simplex virus, an alpha virus, etc., and preferably an adenovirus.

In some embodiments, the vehicle into which the gene encoding STAMP2 is introduced described herein are administered systemically. As used herein, “systemic administration” refers to any means by which the compounds described herein can be made systemically available. In some embodiments, systemic administration encompasses intravenous administration, intraperitoneal administration, intramuscular administration, intracoronary administration, intraarterial administration (e.g., into a carotid artery), intradermal administration, subcutaneous administration, transdermal delivery, intratracheal administration, subcutaneous administration, intraarticular administration, intraventricular administration, inhalation (e. g., aerosol), intracerebral, nasal, naval, oral, intraocular, pulmonary administration, impregnation of a catheter, by suppository and direct injection into a tissue, or systemically absorbed topical or mucosal administration. Mucosal administration includes administration to the respiratory tissue, e.g., by inhalation, nasal drops, ocular drop, etc.; anal or vaginal routes of administration, e.g., by suppositories; and the like. In some embodiments, the compounds described herein are administered intravenously.

The dosage may be adjusted according to factors such as a formulation method, a mode of administration, the age, weight and sex of a subject, a degree of severity of a disease, a diet, an administration time, a route of administration, a secretion rate, and the susceptibility to response. According to one embodiment in which the viral vector is used, the viral vector may be intravenously administered at a dose of 1×10⁸ to 1×10¹¹ plaque-forming units (pfus).

Pharmaceutical formulations suitable for use with the present invention may also include excipients, preservatives, pharmaceutically acceptable carriers and combinations thereof. the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

### Example

### Materials and Methods

Human Pathology Samples

The study included 13 unrelated subjects who had undergone a liver biopsy at the Dong-A University Medical Center (Busan, South Korea). Ten subjects have proved as NAFLD histopathlogically and 3 were control subjects. NAFLD patients consisted of 7 cases of non-alcoholic steatosis, and 3 NASH. Secondary causes of steatosis, including alcohol abuse, liver diseases other than NAFLD, total parenteral nutrition, and the use of drugs known to precipitate steatosis were excluded. Histopathologic diagnoses for NAFLD were underwent according to characteristic histological features. Control subjects were selected among the subjects attending our hospital as living donors for liver transplantation, when their liver biopsy showed only histologically mild steatosis. Their biochemical and imaging findings were not abnormal and they did not have any features of the metabolic syndrome and did not abuse alcohol. The study was approved by the Institutional Review Board of Dong-A University Medical Center (IRB No. 14-158).

Animals

Male C57BL/6 mice were purchased from Samtako, Inc. (Osan, South Korea). The animals were maintained in a temperature-controlled room (22° C.) on a 12:12-h light-dark cycle.

Five-week-old mice were fed an HFD or SD (standard diet) for the indicated durations. The HFD comprised 20% carbohydrate, 20% protein, and 60% fat, for a total of 5.33 kcal per 1 g 17 of diet. All HFD components were purchased from FeedLab (Guri, South Korea). All procedures were approved by the Committee on Animal Investigations at Dong-A University (DIACUC-14-21).

Reagents and Antibodies

Insulin, palmitic acid (PA), oleic acid (OA), stearic acid (SA), fatty acid (FA)-free bovine serum albumin, Oil red O and cycloheximide were purchased from Sigma (St. Louis, Mo., USA). The Lipofectamine 2000 transfection reagent was purchased 1 from Invitrogen (Carlsbad, Calif., USA). The antibodies against IRS1, Akt1/2 and GFP were purchased from Santa Cruz Biotechnology (Santa Cruz, Calif., USA), and antibodies against actin and STAMP2 were obtained from Sigma and Proteintech (Chicago, Ill., USA). Antibodies against phospho-Akt (Ser473), PI3K-p85, phospho-PI3K-p85 (Tyr458), phospho-Tyr, phospho-IRS1(Ser307) were obtained from Cell Signaling (Danvers, Mass., USA).

Cell Culture

HepG2 cells were obtained from the American Type Culture Collection (Manassas, Va., USA). The cells were maintained in Dulbecco's Modified Eagle's Medium (DMEM, Gibco, Grand Island, N.Y., USA), with 10% heat-inactivated fetal bovine serum (FBS) and 1% (v/v) penicillin-streptomycin (PS) at 37° C. in a humid atmosphere of 5% CO2.

Treatment of FFAs

Each FA was dissolved in ethanol and diluted in DMEM containing 0.1% (w/v) FFA-free BSA. In addition, an FFA mixture containing OA (66.7%)/PA (33%) or OA (25%)/PA (42%)/SA (33%) was prepared. FFA-BSA complexes were added to culture plates at known final concentrations of FFAs, which were similar to the physiological ranges of FFAs in fasting and fed states (0.3-1.4 mM). Controls were incubated with equal concentrations of FFA-free BSA containing ethanol.

STAMP2 Knockdown in Mouse Liver

For the siRNA-mediated down-regulation of murine STAMP2, gene-specific siRNA was purchased from Bioneer (Daejeon, South Korea). Control siFVII (supplied by the manufacturer) and siSTAMP2 were complexed (exactly according to the manufacturer's protocol) with Invivofectamine 2.0 Reagent (Invitrogen), which has a high in vivo transfection efficiency in the liver.

Preparation of Adenovirus

The cDNA of murine STAMP2 (GenBank accession no. BC006651) was cloned into a pAdTrack-CMV vector. STAMP2 cDNA was subcloned between KpnI and HindIII of the pAdTrack-CMV expression cassette. Then, a recombinant murine STAMP2 adenovirus was constructed using the AdEasy Adenoviral Vector System. Briefly, the newly constructed pAdTrack-CMV vector (used as control, Ad-Empty) was linearized with PmeI digestion. The linearized vector was then co-transformed into Escherichia coli BJ5183, along with the pAdEasy1 vector. All viruses were propagated in 293 cells, purified by CsCl density purification, dissolved in 1×HBSS (Invitrogen), and stored at −70° C. until used.

In Vivo Delivery of siSTAMP2 and Adenoviral STAMP2

siRNA-Invivofectamine 2.0 complexes (100 μL of 0.7 mg/mL relative to siRNA) was injected through the tail veins of mice fed an HFD for 10 weeks. siSTAMP-Invivofectamine 2.0 complex-injected mice fed an SD or siFVII-Invivofectamine 2.0 complex-injected mice fed an SD or HFD were used as experimental controls. Ten days after the siRNA injection, various assays were performed to evaluate the effect of liver-specific STAMP2 deletion on hepatic steatosis. The STAMP2-expressing adenovirus of 1×1010 plaque-forming units (PFUs) or control vehicle virus was injected into the tail veins of mice fed an HFD or SD for 15 weeks. Biochemical and histological analyses were performed 7 days after the injection.

Histology Staining

Liver tissues were fixed in 10% neutral buffered formalin and embedded in paraffin. Four micrometer sections were prepared and stained with hematoxylin and eosin. The morphology of the liver tissue was photographed using an Aperio ScanScope (Aperio Technologies, Vista, Calif., USA).

Immunohistochemical Staining

The sections from the mice were incubated overnight with primary rabbit polyclonal STAMP2 antibody and then with a matching biotinylated secondary antibody for 30 min at 37° C. The negative controls did not have the primary antibody. The stained sections were developed with diaminobenzidine and counterstained with hematoxylin. Immunohistochemical staining for human sample was performed using the DiscoveryXT automated immunohistochemistry stainer (Ventana Medical Systems, Inc., Tucson, Ariz., USA). Slides were incubated with anti-STAMP2 antibody for 24 min. Secondary antibody of Dako REAL™ Envision™ anti-abbit/Mouse HRP (Dako, Denmark) was treated for 8 min, then slides were incubated in DAB+H2O2 substrate. The results were viewed using an Aperio ScanScope.

Oil Red O Staining

Cells were washed twice in PBS and fixed for 1 h with 10% (w/v) formaldehyde in PBS. After two washes in 60% isopropyl alcohol, the cells were stained for 30 min in freshly diluted Oil Red O solution. Then, the stain was removed, and the cells were washed 4 times in water. After adding 100% 2-propanol at 500 nm, the absorbance of the eluted Oil Red O was measured in a spectrophotometer.

Plasma Glucose Concentrations and Tolerance Tests for Glucose and Insulin

Intraperitoneal glucose tolerance tests (GTTs) and insulin tolerance tests (ITTs) were performed after the mice were fasted for 16 h. Plasma glucose concentrations were measured in tail blood using a GlucoDr Blood Glucose Test Strip (Hasuco, Seoul, South Korea) prior to and 15, 30, 60, 90 and 120 minutes after intraperitoneally injecting a bolus of glucose (1 mg/g) for the GTT and at the same time points after intraperitoneally injecting 0.75 U/kg body weight insulin for the ITT.

Analysis of Plasma Insulin and Metabolites

Plasma insulin was measured with a mouse insulin ELISA kit (Shibayagi, Gunma, Japan). Plasma total cholesterol (TC), triglyceride (TG), and nonesterified fatty acids (NEFA) were measured using an enzymatic, colorimetric test kit (Asan Pharmaceutical Co., Seoul, Korea).

Luciferase Assay

HepG2 cells were plated in a 24-well culture plate and transfected with 1 a reporter vector (0.2 μg) together with each indicated expression plasmid using Lipofectamine 2000 (Invitrogen), according to the manufacturer's instructions. The luciferase activities were measured using the Dual Luciferase Reporter Assay System (Promega, Madison, Wis., USA), according to the manufacturer's instructions. Firefly luciferase activities were standardized to Renilla activities.

RNA Isolation and RT-PCR

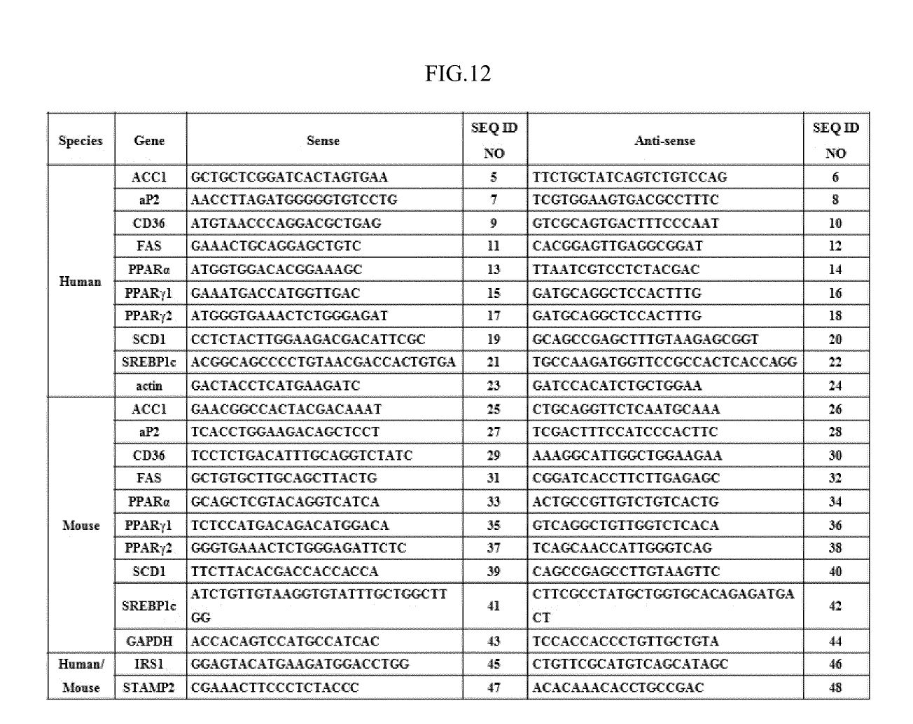

Total RNA was prepared from cell lines or tissues using TRIzol reagent (Invitrogen), according to the manufacturer's instructions. Then, 5 μg of total RNA was converted into single-stranded cDNA using MMLV reverse transcriptase (Takara, Tokyo, Japan), with random hexamer primers. A one-tenth aliquot of the cDNA was subjected to PCR amplification using gene-specific primers (FIG. 12). The RT-PCR bands were quantified and normalized relative to the β-actin mRNA control band with ImageJ version 1.48q (National Institutes of Health imaging software).

Western Blot Analysis

Cells were washed twice with ice-cold PBS, resuspended in 100 μL ice-cold RIPA buffer and incubated at 4° C. for 30 min. Lysates were centrifuged at 13,000 rpm for 15 min at 4° C. Equal amounts of proteins were subjected to 7.5-15% sodium dodecyl sulfate polyacrylamide gel electrophoresis. The proteins were transferred to a nitrocellulose membrane (Amersham Pharmacia Biotech, Piscataway, N.J., USA) and reacted with each antibody Immunostaining with antibodies was performed using the Super Signal West Pico (Thermo 1 Scientific, Hudson, N.H., USA) enhanced chemiluminescence substrate and detected with LAS-3000 Plus (Fuji Photo Film, Tokyo, Japan). Quantification and normalization to GAPDH or actin control bands using ImageJ version 1.48q.

Statistical Analysis

At least three independent experiments were conducted. The results are expressed as the means±standard deviation (SD). The statistical significance of the differences was primarily determined using the Mann-Whitney U test. Pairwise comparison of the insulin-induced suppression of the promoter activity of PECK and G6Pase between treatments was determined using the Ancova test. P<0.05 indicated statistical significance.

Results

STAMP2 Expression is Markedly Reduced in the Livers Obtained from Human NAFLD Patients and NAFLD Model Mice

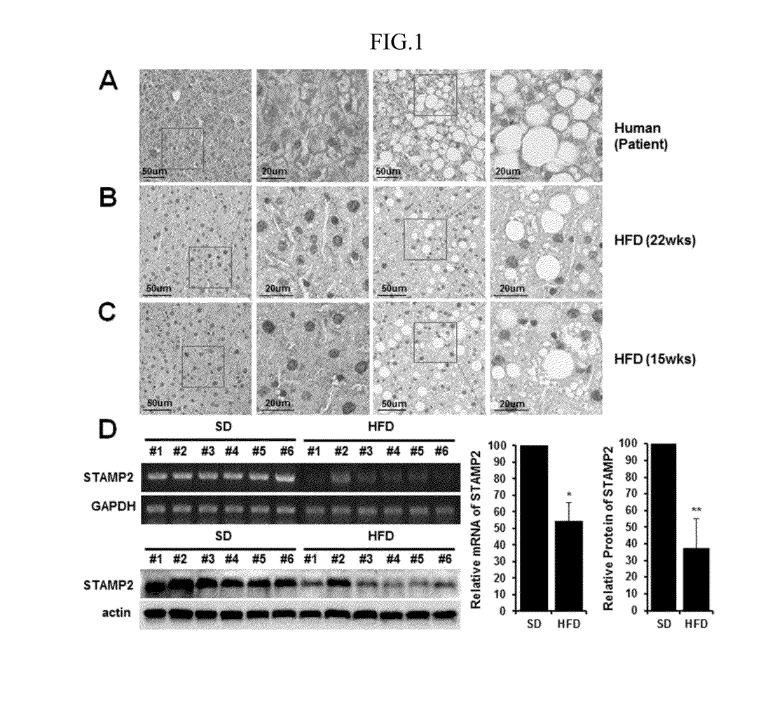

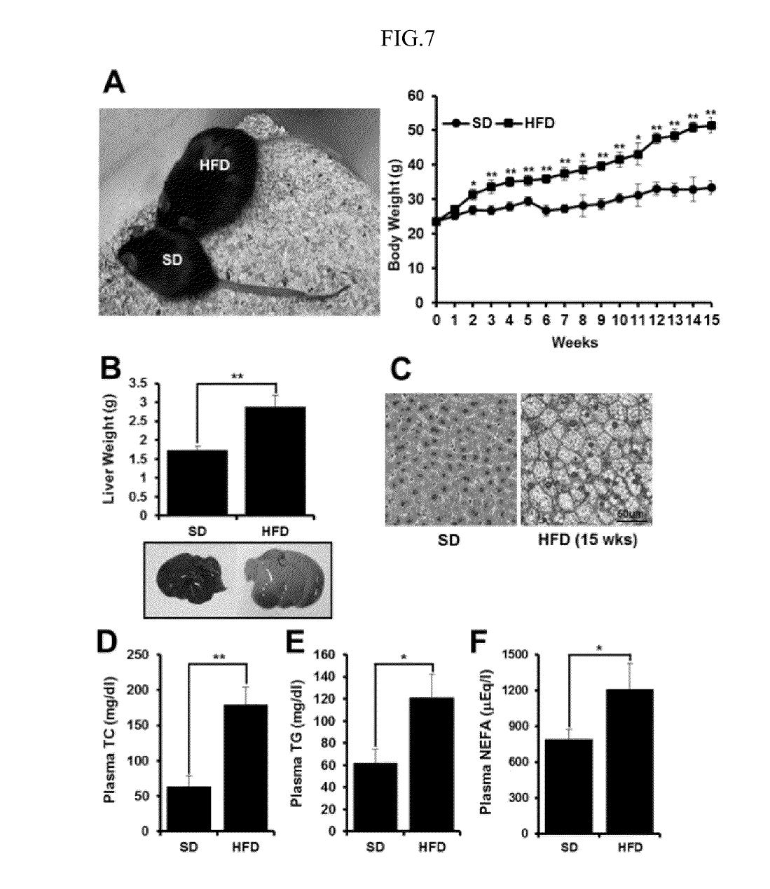

Immuunohistochemistry demonstrated that the hepatic expression of STAMP2 is markedly reduced in liver tissue from the non-alcoholic steatosis patients(left) compared to the non-NAFLD controls(right) (A of FIG. 1). Liver from NASH patients revealed meager reduced expression (data not shown). We performed additional immunohistochemistry on the liver sections from NAFLD C57BL/6 mice that had been preserved for the prior study, in which they were fed an HFD for 22 weeks. We also observed that the hepatic expression of STAMP2 is markedly reduced in mice fed an HFD compared with mice fed an SD (B of FIG. 1). Henceforth, all in vivo data were obtained from an HFD-induced NAFLD model that was specifically designed for this study in which HFD induces hepatic steatosis and insulin resistance (FIGS. 7&8). Immunohistochemistry and western blot analysis showed that the hepatic expression of STAMP2 was markedly reduced in the mice fed an HFD compared with the mice fed an SD (C and D of FIG. 1). These findings suggest that STAMP2 plays a role in preventing the development of NAFLD.

Liver Specific Deletion of STAMP2 Accelerates Hepatic Steatosis and Insulin Resistance in HFD-Induced NAFLD Mice

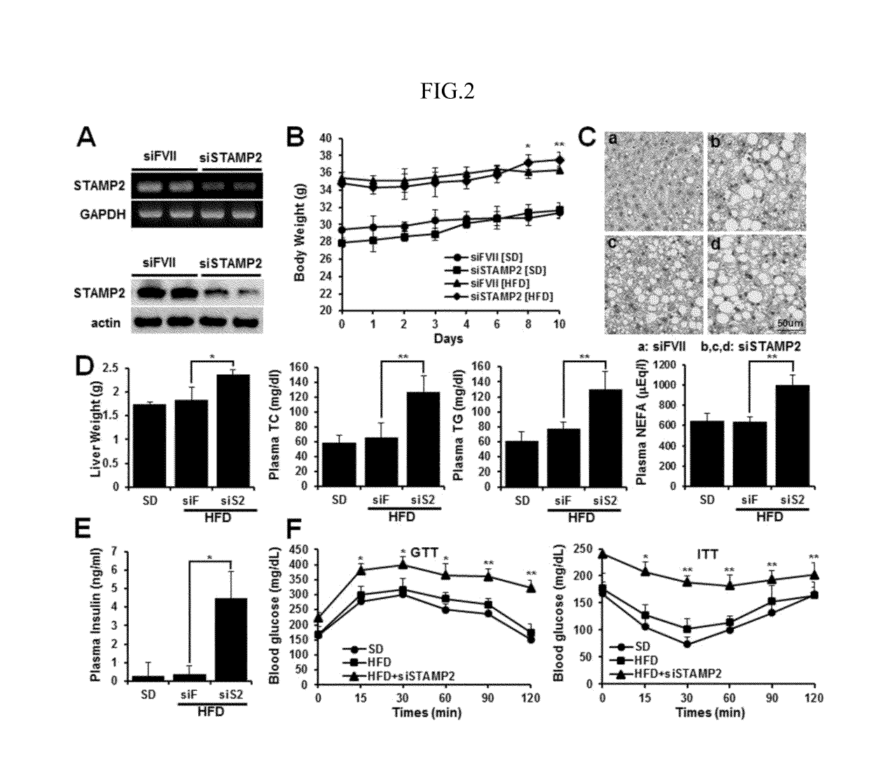

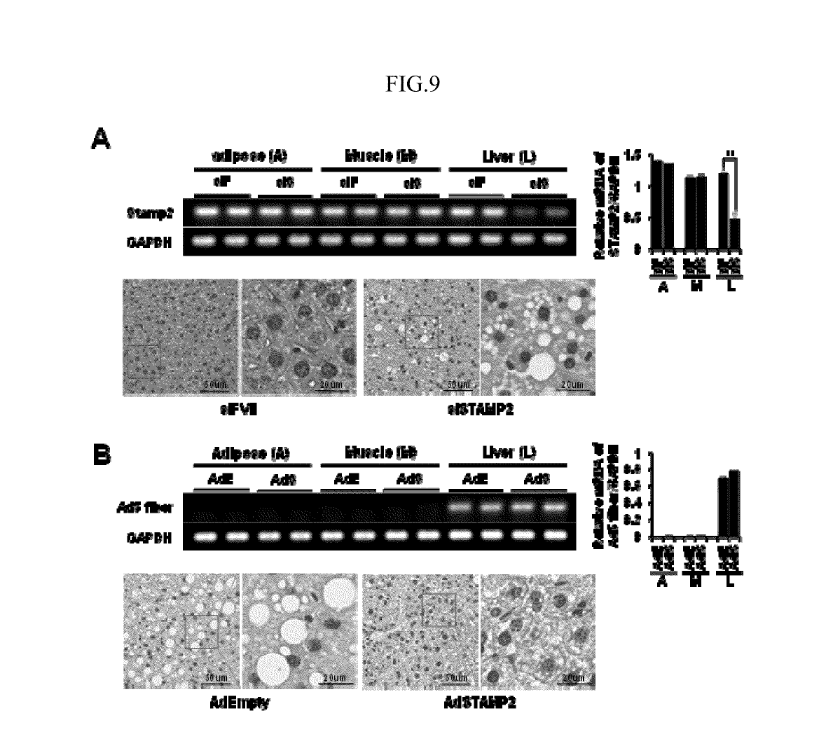

To prove that STAMP2 prevents the development of NAFLD, we delivered siSTAMP2 to the livers of mice fed an HFD for 10 weeks. siSTAMP2 efficiently downregulated the STAMP2 gene and protein expression in the liver but not in the adipose tissue and muscular tissues (A of FIG. 2 and A of FIG. 9) Importantly, 10 days after the injection, the liver-specific deletion of STAMP2 induced hepatic steatosis (B-D of FIG. 2) and insulin resistance (E and F of FIG. 2), while at the same time point, the experimental control mice exhibited neither hepatic steatosis nor insulin resistance.

Adenoviral Overexpression of STAMP2 Improves Hepatic Steatosis in HFD-Induced NAFLD Mice

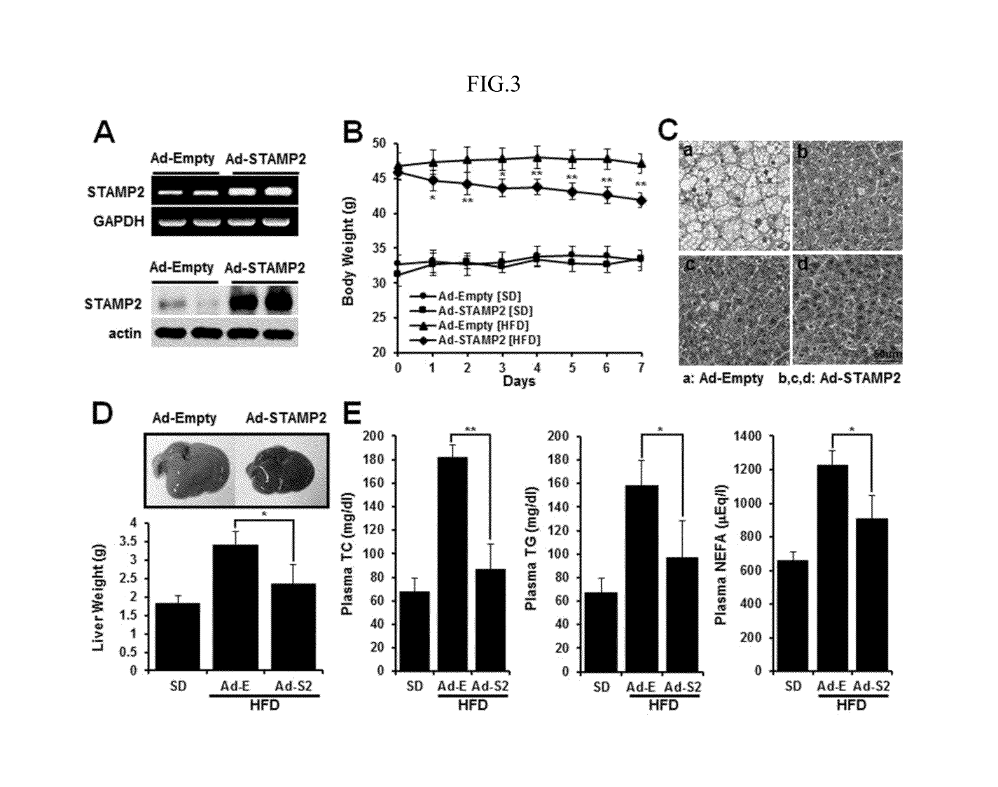

Our data suggested that STAMP2 represents a suitable target for intervention targeting NAFLD; therefore, next, we examined the effect of overexpressed STAMP2 in HFD-induced NAFLD. Ad-STAMP2 efficiently increased STAMP2 gene and protein expression in the liver but not in the adipose and muscular tissues (A of FIG. 3 and B of FIG. 9). Notably, Ad-STAMP2 reversed various manifestations of hepatic steatosis, including body and liver weight gain, increased vacuolization/lipid accumulation and increased plasma lipid levels (B-E of FIG. 3).

STAMP2 Reduces In Vitro FFA-Induced Lipid Accumulation

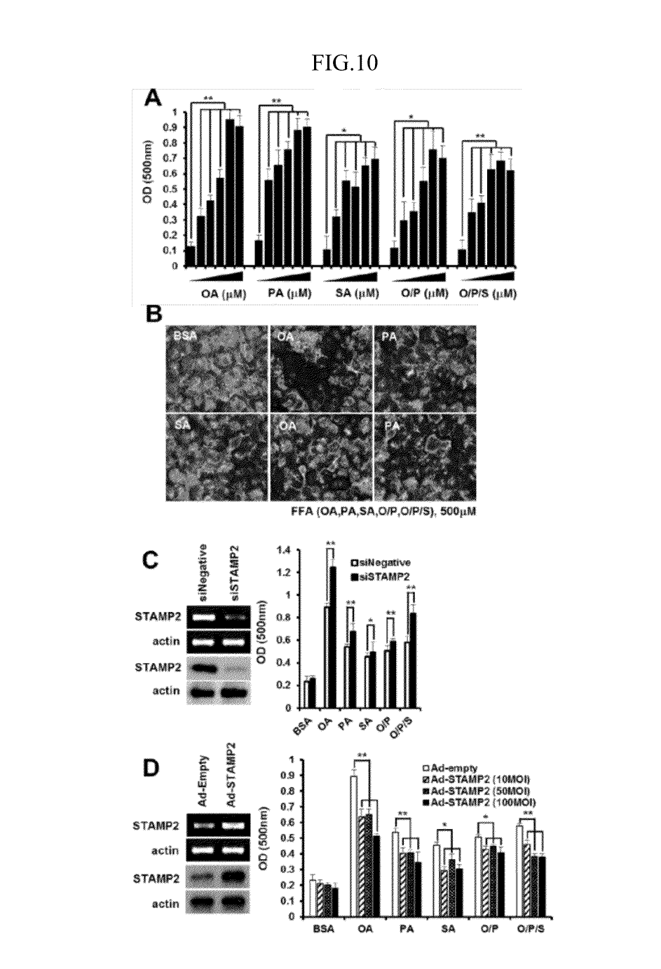

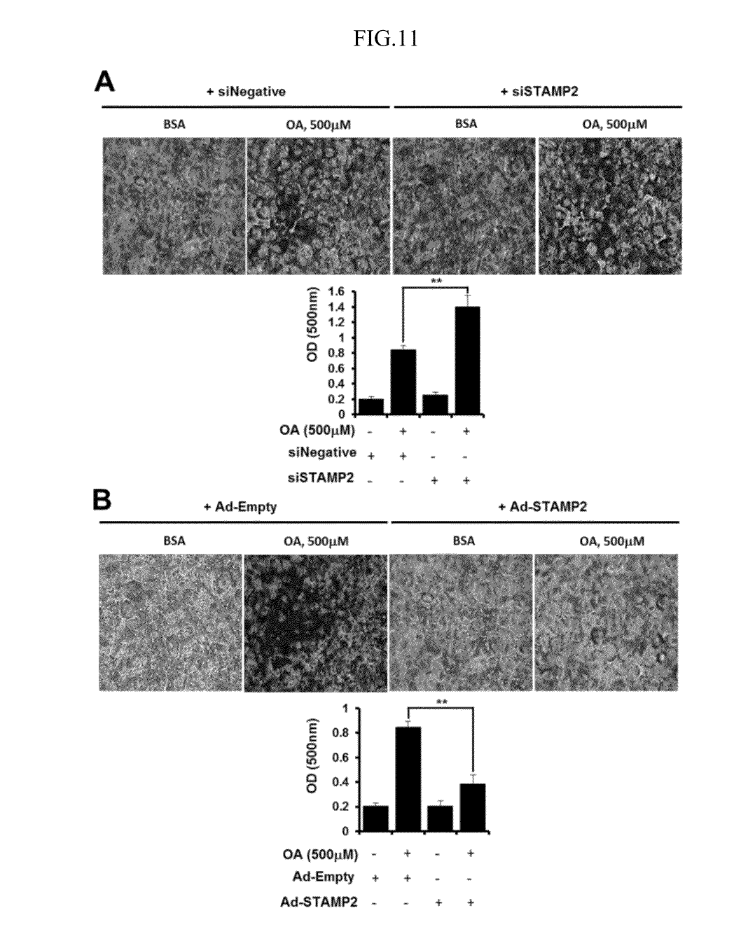

Next, we investigated whether STAMP2 regulates FFA-induced lipid accumulation in hepatocytes in vitro. We observed that all three FFAs and FFA mixtures induced lipid accumulation in HepG2 cells in a dose dependent manner (A and B of FIG. 10). Silencing of the STAMP2 gene significantly augmented FFA-induced lipid accumulation (C of FIG. 10). Conversely, overexpression of STAMP2 significantly reduced the FFA-induced lipid accumulation (D of FIG. 10). Among three FFAs, the effect of STAMP2 was most prominent in hepatocytes treated with oleic acid (OA); therefore, further study of the additional molecular mechanisms through which hepatic STAMP2 reduces lipid imbalance was performed using this OA-induced NAFLD in vitro model (FIG. 11).

Hepatic STAMP2 Downregulates Lipogenic and Adipogenic Factors In Vivo and In Vitro

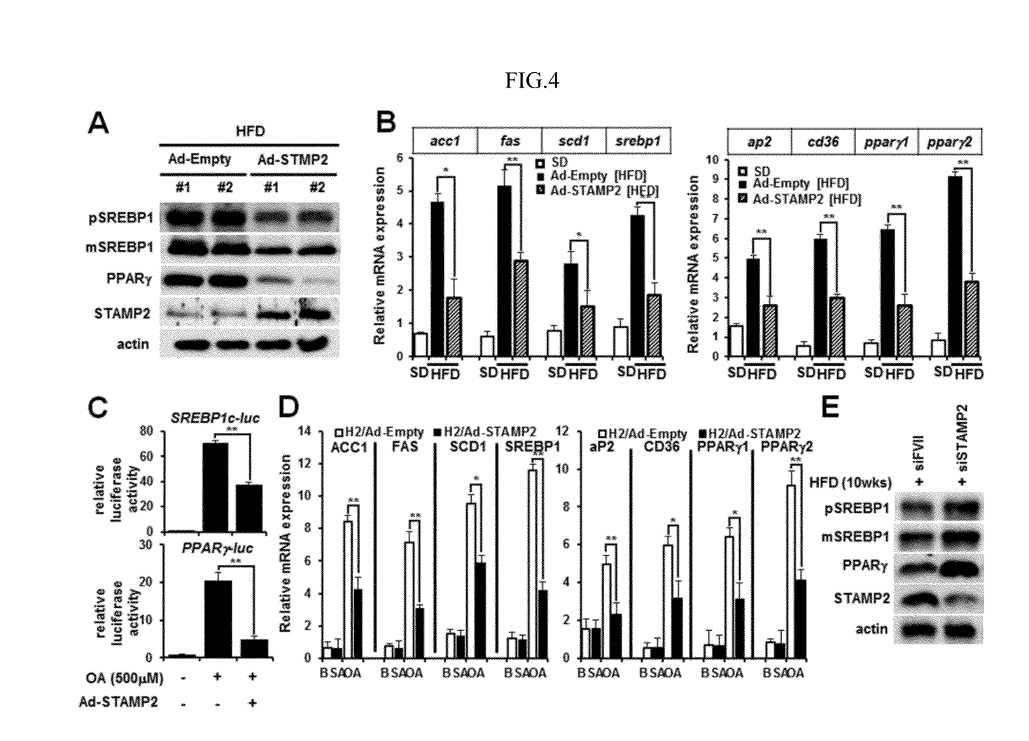

Then, we examined whether hepatic STAMP2 modulated the expression and activity of SREBP1 and PPARγ which crucially mediates the development of hepatic steatosis. Ad-STAMP2 counteracted the HFD-induced upregulation of SREBP1 and PPARγ proteins (A of FIG. 4), as well as of their target genes (B of FIG. 4) in an HFD-induced NAFLD 1 model. In addition, Ad-STAMP2 counteracted the OA-induced increase of their promoter activity (C of FIG. 4) and mRNA expression (D of FIG. 4) in hepatocytes in vitro. The liver-specific deletion of STAMP2 markedly augmented HFD-induced increase of SREBP1 and PPARγ proteins level (E of FIG. 4). These results indicate that hepatic STAMP2 improves hepatic lipid accumulation by downregulating lipogenic and adipogenic factors.

Adenoviral Overexpression of STAMP2 Improves In Vitro and In Vivo Insulin Sensitivity and Glucose Metabolism

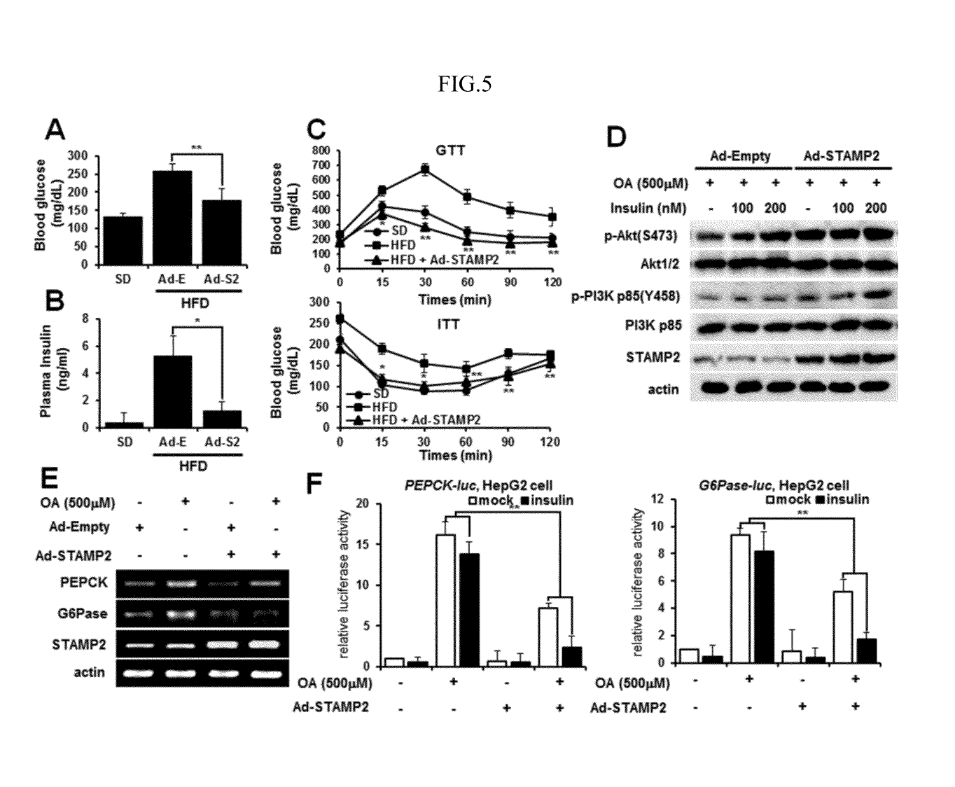

Ad-STAMP2 attenuated the HFD-induced whole body insulin resistance in vivo (A-C of FIG. 5). Furthermore, Ad-STAMP2 attenuated the OA-induced insulin resistance in vitro (D-F of FIG. 5). Adenoviral STAMP2 augmented the insulin-induced up-regulation of p-Akt(Ser473) and p-PI3K p85(Tyr458) in an OA-induced NAFLD in vitro model (D of FIG. 5). Because insulin reduces gluconeogenesis through the specific transcriptional inhibition of PEPCK and G6Pase, we attempted to determine whether Ad-STAMP2 improved OA-induced attenuation of insulin signaling through suppressing PEPCK and G6Pase expression. Although OA increased the expression levels of PEPCK and G6Pase mRNAs, Ad-STAMP2 reversed these OA-induced increased expression levels (E of FIG. 5). The luciferase assay revealed that Ad-STAMP2 enhanced insulin-induced suppression of the promoter activity of these genes in OA-treated HepG2 cells (F of FIG. 5). These findings indicate that STAMP2 improves insulin signaling, resulting in the inhibition of gluconeogenesis.

Hepatic STAMP2 Prevents IRS1 Degradation

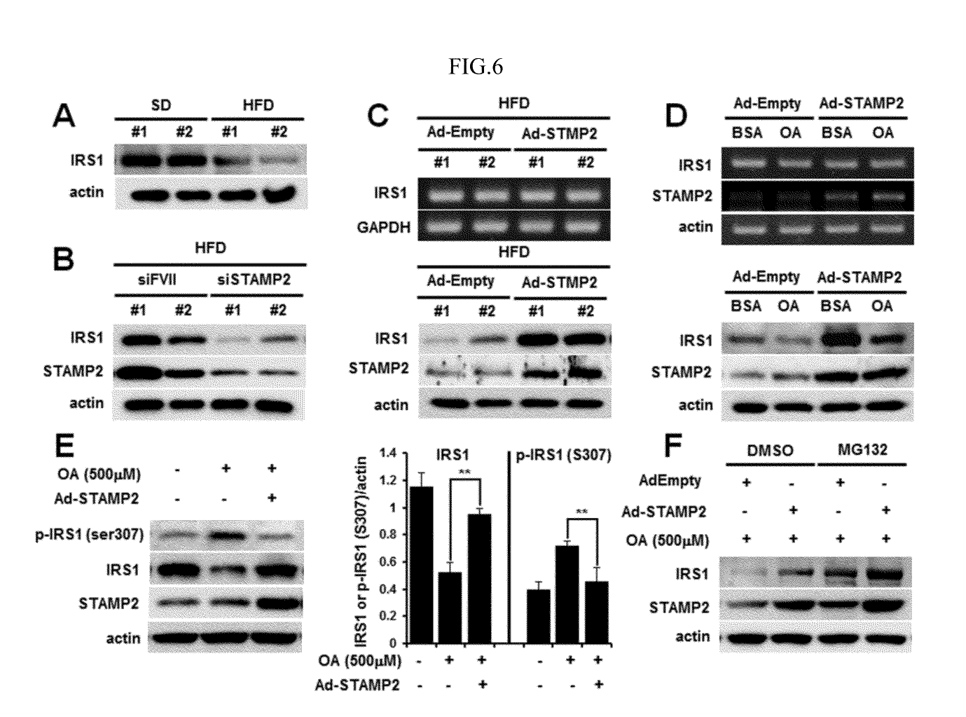

Next, we further investigated the molecular mechanism through which STAMP2 improves insulin resistance in the liver. The IRS1 protein level was markedly reduced in HFD-induced NAFLD mice (A of FIG. 6) and liver specific STAMP2 deleted mice (B of FIG. 6). Ad-STAMP2 markedly reversed not only HFD-induced downregulation of the IRS1 protein level in vivo but also OA-induced downregulation of the IRS1 protein level in vitro (C and D of FIG. 6). However, the mRNA level of IRS1 did not change in vivo or in vitro (C and D of FIG. 6). OA increased the phosphorylation of Ser307 on IRS1, which has been correlated with IRS1 degradation. Noticebly, Ad-STAMP2 reversed the OA-induced increase of phosphorylation of Ser307 on IRS1 (E of FIG. 6). In addition, the protein level of IRS1 accumulated by treatment with proteasome inhibitor MG132 (F of FIG. 6). These results indicate that hepatic STAMP2 regulates the IRS1 protein level through post-translational control.

1. A method for treating or preventing a diet-induced non-alcoholic fatty liver disease, comprising promoting the expression of STAMP2 (Six-transmembrane protein of prostate 2) in a liver cell by administering a vehicle, into which a gene encoding STAMP2 is introduced, to a subject.

2. (canceled)

3. The method according to claim 1, wherein the vehicle is an adenovirus.

4. The method according to claim 1, wherein the administration is intravenous administration.

5. The method according to claim 1, wherein the vehicle is administered at a dose of 1×10⁸ to 1×10¹¹ plaque-forming units (pfus).

6. The method according to claim 1, by the promotion of the expression, the expression of at least one protein selected from the group consisting of ACC1, FAS, SCD1, SREBP1, aP2, CD36, and PPARγ in liver cells is inhibited.

7. The method according to claim 1, by the promotion of the expression, the degradation of IRS1 in the liver cells is inhibited.

8. The method according to claim 1, wherein the non-alcoholic fatty liver disease is selected from the group consisting of non-alcoholic simple steatosis, non-alcoholic steatohepatitis, non-alcoholic steatohepatitis with liver fibrosis, non-alcoholic steatohepatitis with cirrhosis, and non-alcoholic steatohepatitis with cirrhosis and hepatocellular carcinoma.

9. A method for treating a subject having a diet-induced non-alcoholic fatty liver disease, comprising administering an effective amount of a vehicle, into which a gene encoding STAMP2 is introduced, to the subject.

10. The method according to claim 9, wherein the vehicle is an adenovirus.

11. The method according to claim 9, wherein the administration is intravenous administration.

12. The method according to claim 9, wherein the effective amount of the vehicle is in a range of 1×10⁸ to 1×10¹¹ plaque-forming units (pfus).

13. The method according to claim 9, by the administration, the expression of at least one protein selected from the group consisting of ACC1, FAS, SCD1, SREBP1, aP2, CD36, and PPARγ in liver cells of the subject is inhibited.

14. The method according to claim 9, further comprising a step of inhibiting the degradation of IRS1 in the liver cells after the step of promoting the expression of STAMP2.

15. The method according to claim 9, wherein the non-alcoholic fatty liver disease is selected from the group consisting of non-alcoholic simple steatosis, non-alcoholic steatohepatitis, non-alcoholic steatohepatitis with liver fibrosis, non-alcoholic steatohepatitis with cirrhosis, and non-alcoholic steatohepatitis with cirrhosis and hepatocellular carcinoma.

16-19. (canceled)

20. The method of claim 1, wherein the method is for treating the diet-induced non-alcoholic fatty liver disease, and the subject has the diet-induced non-alcoholic fatty liver disease.
